(19) United States
(12) Patent Application Publication
(10) Pub. No.: US 2007/0275389 A1
(43) Pub. Date: Nov. 29, 2007

(54) Array design facilitated by consideration of hybridization kinetics

(76) Inventors: Anniek De Witte, Palo Alto, CA (US); James M. Minor, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/441,071

(22) Filed: May 24, 2006

(51) Int. Cl. C12Q 1/68; G06F 19/00

(52) U.S. Cl. 435/6; 702/20

(57) ABSTRACT

Methods, systems and computer readable media for selecting probes for design of a chemical array. A first set of candidate probes is provided for hybridization with a sample at a first hybridization stringency and a second set of candidate probes identical to the first set is provided for hybridization with the sample at a second hybridization stringency. After hybridizing the first set with the sample at the first hybridization stringency and the second set with the sample at the second hybridization stringency higher than the first hybridization stringency, the relative change in signal extracted from a probe in the first set relative to the same probe in the second set is calculated, and this calculation is carried out for each of a plurality of (up to and including all) other probes in the first set and same probes in the second set, respectively. At least the probe having the highest calculated relative change in signal between the first and second hybridization stringencies is eliminated as a candidate for use in the array design. Methods, systems and computer readable media for identifying relative degrees of non-specific binding of probes hybridized with a sample. A first set of probes is provided for hybridization with a sample at a first hybridization stringency and a second set of probes identical to the first set is provided for hybridization with the sample at a second hybridization stringency. After hybridizing the first set with the sample at a first hybridization stringency and hybridizing the second set with the sample at a second hybridization stringency higher than the first hybridization stringency, the relative change in signal extracted from a probe in the first set relative to the same probe in the second set is calculated, and this calculation is repeated for each of a plurality (up to, and including all) of other probes in the first set and same probes in the second set, respectively. The probes are then ranked by degree of non-specific binding, wherein the probe having the highest calculated relative change in signal between the first and second hybridization stringencies is ranked highest.

### CROSS-REFERENCE

This application is related to Application Serial No. (application Ser. No. not yet assigned, Attorney's Docket No. 10051786-1) filed concurrently herewith and titled “Programmed Changed in Hybridization Conditions to Improve Probe Quality”, which is hereby incorporated herein, in its entirety, by reference thereto.

### BACKGROUND OF THE INVENTION

Arrays of binding agents or probes, such as polypeptide and nucleic acids, have become an increasingly important tool in the biotechnology industry and related fields. These binding agent arrays, in which a plurality of probes are positioned on a solid support surface in the form of an array or pattern, find use in a variety of different fields, e.g., genomics (in sequencing by hybridization, SNP detection, differential gene expression analysis, CGH analysis, location analysis, identification of novel genes, gene mapping, finger printing, etc.) and proteomics.

In using such arrays, the surface-bound probes are contacted with molecules or analytes of interest, i.e., targets, in a sample. Targets in the sample bind to the complementary probes on the substrate to form a binding complex. The pattern of binding of the targets to the probe features or spots on the substrate produces a pattern on the surface of the substrate and provides desired information about the sample. In most instances, the targets are labeled with a detectable label or reporter such as a fluorescent label, chemiluminescent label or radioactive label. The resultant binding interaction or complexes of binding pairs are then detected and read or interrogated, for example, by optical means, although other methods may also be used depending on the detectable label employed. For example, laser light may be used to excite fluorescent labels bound to a target, generating a signal only in those spots on the substrate that have a target, and thus a fluorescent label, bound to a probe molecule. This pattern may then be digitally scanned for computer analysis.

Generally, in discovering or designing probes to be used in an array, a nucleic acid sequence is selected based on the particular gene or genetic locus of interest, where the nucleic acid sequence may be as great as about 60 or more nucleotides in length, or as small as about 25 nucleotides in length or less. From the nucleic acid sequence, probes are synthesized according to various nucleic acid sequence regions, i.e., subsequences of the nucleic acid sequence and are associated with a substrate to produce a nucleic acid array. As described above, a detectably labeled sample is contacted with the array, where targets in the sample bind to complementary probe sequences of the array.

As is apparent, a step in designing arrays is the selection of a specific probe or mixture of probes that may be used in the array and which increase the chances of binding with a specific target in a sample, while at the same time reducing the time and expense involved in probe discovery and design. In practice, designing an optimized array typically involves iterating the array design one or more times to replace probes that are found to be undesirable for detecting targets of interest, either due to poor signal quality and/or cross-hybridization with sequences other than the targets of interest. Such iterations are costly and time consuming.

For example, conventional probe design may be performed experimentally or computationally (i.e., in silico), where in many instances it is performed computationally. Accordingly, probe design usually involves taking subsequences of a nucleic acid and filtering them based on certain computationally determined values such as melting temperature, self structure, homology, etc., to attempt to predict which subsequences will generate probes that will provide good signal and/or will not cross-hybridize. The subsequences that remain after the filtering process are selected to generate probes to be used in nucleic acid arrays. Thus, a database of probe characteristics may be provided and stored, from which to select probes for an array design based on characteristics, such as those described above, which are desirable for the array being designed.

While attempts have been made to predict which probes will provide the best results in an array assay, such attempts are not completely satisfactory as probes selected using these methods are often still found to be undesirable for one or both of the above-described reasons. In other words, some probes will still fail or give false results as the computational techniques used to filter and select the probes are not precise predictors. Accordingly, as mentioned above, typically an array design must be iterated a number of times in order to filter out all the undesirable probes from the array. Furthermore, such attempts often characterize probes after they have been synthesized, that is after time and expense have already been invested.

There is continued interest in the development of new methods, including empirical methods, and devices for producing arrays of nucleic acid probes that provide strong signal and do not cross-hybridize with sequences other than targets of interest.

### SUMMARY OF THE INVENTION

Methods, systems and computer readable media are provided for selecting probes for design of a chemical array. A first set of candidate probes is provided for hybridization with a sample at a first hybridization stringency and a second set of candidate probes identical to the first set is provided for hybridization with the sample at a second hybridization stringency. After hybridizing the first set with the sample at a first hybridization stringency, and hybridizing the second set with the sample at a second hybridization stringency higher than the first hybridization stringency, the relative change in signal extracted from a probe in the first set relative to the same probe in the second set is calculated, and this calculation is repeated for each of a plurality of other probes in the first set and same probes in the second set, respectively. At least the probe having the highest calculated relative change in signal between the first and second hybridization stringencies is eliminated as a candidate for use in the array being designed.

Methods, systems and computer readable media are provided for identifying relative degrees of non-specific binding of probes hybridized with a sample. A first set of probes is provided for hybridization with a sample at a first hybridization stringency and a second set of probes identical to the first set is provided for hybridization with the sample at a second hybridization stringency. After hybridizing the first set with the sample at a first hybridization stringency and hybridizing the second set with the sample at a second hybridization stringency higher than the first hybridization stringency, the relative change in signal extracted from a probe in the first set relative to the same probe in the second set is calculated and the calculation is repeated for each of a plurality of other probes in the first set and same probes in the second set, respectively. The probes are then ranked by degree of non-specific binding, wherein the probe having the highest calculated relative change in signal between the first and second hybridization stringencies is ranked highest.

Arrays for carrying out the methods disclosed herein are also provided.

Kits for carrying out the methods disclosed herein are also provided.

These and other features of the invention will become apparent to those persons skilled in the art upon reading the details of the methods, systems and computer readable media as more fully described below.

### BRIEF DESCRIPTION OF THE DRAWINGS

FIG. 1 shows an exemplary substrate carrying an array, such as may be feature extracted by a feature extraction system to provide feature extraction output data.

FIG. 2 shows an enlarged view of a portion of FIG. 1 showing spots or features.

FIG. 3 illustrates events that may be carried out to estimate probe performance for selection of probes exhibiting the best performance for an array design.

FIG. 4 is a schematic illustration of a typical computer system that may be used to perform procedures described herein.

FIGS. 5A-5C show plots of a bivariate fit of LogRatio70 values versus scores for the same.

### DETAILED DESCRIPTION OF THE INVENTION

Before the present methods, systems and computer readable media are described, it is to be understood that this invention is not limited to particular genes, genomes, methods, method steps, statistical methods, hardware or software described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a probe” includes a plurality of such probes and reference to “the sample” includes reference to one or more samples and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFININTIONS

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides.. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source.

An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups).

A nucleotide “Probe” means a nucleotide which hybridizes in a specific manner to a nucleotide target sequence (e.g. a consensus region or an expressed transcript of a gene of interest).

A “chemical array”, “microarray”, “bioarray” or “array”, unless a contrary intention appears, includes any one-, two- or three-dimensional arrangement of addressable regions bearing a particular chemical moiety or moieties associated with that region. A microarray is “addressable” in that it has multiple regions of moieties such that a region at a particular predetermined location on the microarray will detect a particular target or class of targets (although a feature may incidentally detect non-targets of that feature). Array features are typically, but need not be, separated by intervening spaces. In the case of an array, the “target” will be referenced as a moiety in a mobile phase, to be detected by probes, which are bound to the substrate at the various regions. However, either of the “target” or “target probes” may be the one, which is to be evaluated by the other.

Methods to fabricate arrays are described in detail in U.S. Pat. Nos. 6,242,266; 6,232,072; 6,180,351; 6,171,797 and 6,323,043. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Following receipt by a user, an array will typically be exposed to a sample and then read. Reading of an array may be accomplished by illuminating the array and reading the location and intensity of resulting fluorescence at multiple regions on each feature of the array. For example, a scanner may be used for this purpose is the AGILENT MICROARRAY SCANNER manufactured by Agilent Technologies, Palo, Alto, Calif. or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. Scanning typically produces a scanned image of the array which may be directly inputted to a feature extraction system for direct processing and/or saved in a computer storage device for subsequent processing. However, arrays may be read by any other methods or apparatus than the foregoing, other reading methods including other optical techniques or electrical techniques (where each feature is provided with an electrode to detect bonding at that feature in a manner disclosed in U.S. Pat. Nos. 6,251,685, 6,221,583 and elsewhere). In any case, detection is made for the purpose of identifying and quantifying of the particular target(s) bonded (i.e., hybridized) to a particular probe.

An array is “addressable” when it has multiple.regions of different moieties, i.e., features (e.g., each made up of different oligonucleotide sequences) such that a region (i.e., a “feature” or “spot” of the array) at a particular predetermined location (i.e., an “address”) on the array will detect a particular solution phase nucleic acid sequence. Array features are typically, but need not be, separated by intervening spaces.

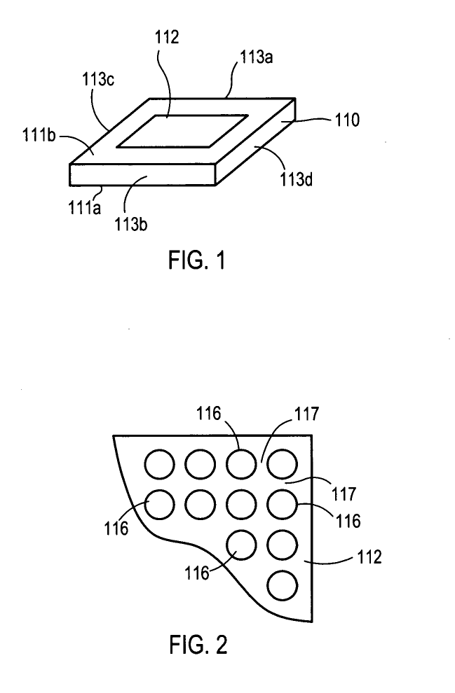

An exemplary array is shown in FIGS. 1-2, where the array shown in this representative embodiment includes a contiguous planar substrate 110 carrying an array 112 disposed on a surface 111b of substrate 110. It will be appreciated though, that more than one array (any of which are the same or different) may be present on surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the surface 111b, with regions of the surface 111b adjacent the opposed sides 113c, 113d and leading end 113a and trailing end 113b of slide 110, not being covered by any array 112. A surface 111a of the slide 110 typically does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of oligomers, e.g., in the form of polynucleotides, and specifically oligonucleotides. As mentioned above, all of the features 116 may be different, or some or all could be the same. The interfeature areas 117 could be of various sizes and configurations. Each feature carries a predetermined oligomer such as a predetermined polynucleotide (which includes the possibility of mixtures of polynucleotides). It will be understood that there may be a linker molecule (not shown) of any known types between the surface 111b and the first nucleotide.

Substrate 110 may carry on surface 111a, an identification code, e.g., in the form of bar code (not shown) or the like printed on a substrate in the form of a paper or plastic label attached by adhesive or any convenient means. The identification code contains information relating to array 112, where such information may include, but is not limited to, an identification of array 112, i.e., layout information relating to the array(s), etc.

In the case of an array in the context of the present application, the “target” may be referenced as a moiety in a mobile phase (typically fluid), to be detected by “probes” which are bound to the substrate at the various regions.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

A “design file” is typically provided by an array manufacturer and is a file that embodies all the information that the array designer from the array manufacturer considered to be pertinent to array interpretation. For example, Agilent Technologies supplies its array users with a design file written in the XML language that describes the geometry as well as the biological content of a particular array.

A “grid template” or “design pattern” is a description of relative placement of features, with annotation. A grid template or design pattern can be generated from parsing a design file and can be saved/stored on a computer storage device. A grid template has basic grid information from the design file that it was generated from, which information may include, for example, the number of rows in the array from which the grid template was generated, the number of columns in the array from which the grid template was generated, column spacings, subgrid row and column numbers, if applicable, spacings between subgrids, number of arrays/hybridizations on a slide, etc. An alternative way of creating a grid template is by using an interactive grid mode provided by the system, which also provides the ability to add further information, for example, such as subgrid relative spacings, rotation and skew information, etc.

“Image processing” refers to processing of an electronic image file representing a slide containing at least one array, which is typically, but not necessarily in TIFF format, wherein processing is carried out to find a grid that fits the features of the array, e.g., to find individual spot/feature centroids, spot/feature radii, etc. Image processing may even include processing signals from the located features to determine mean or median signals from each feature and may further include associated statistical processing. At the end of an image processing step, a user has all the information that can be gathered from the image.

“Post processing” or “post processing/data analysis”, sometimes just referred to as “data analysis” refers to processing signals from the located features, obtained from the image processing, to extract more information about each feature. Post processing may include but is not limited to various background level subtraction algorithms, dye normalization processing, finding ratios, and other processes known in the art.

“Feature extraction” may refer to image processing and/or post processing, or just to image processing. An extraction refers to the information gained from image processing and/or post processing a single array.

“Stringency” is a term used in hybridization experiments to denote the degree of homology between the probe and the target hybridized thereto. The higher the stringency, the higher percent homology between the probe and target. Hybridization stringency may be effected by a change in temperature and/or chemical process steps such as the amounts of salts and/or formamide in the hybridization solution during a hybridization process.

“in silico metrics” are those metrics that can be calculated in the absence of any experimental data. They can be derived from the probe sequences of the probes themselves and from the sequences of the genome or the transcriptome of the respective organism. in silico metrics can be used for each candidate probe that are calculated from the sequences directly, using the known laws of physics or chemistry, such as those related to thermodynamics. These metrics include (but are not limited to): duplex melting temperature (Tₘ or DuplexTm) between a probe and its complementary sequence; the probes' maximal subsequence duplex melting temperature, which we define as the maximal Tₘ for any subsequence of length M within a longer sequence of length N. (MaxSubSeqTm); hairpin thermodynamics of the probe, such as expressed in terms of its hairpin melting temperature, or Gibbs Free energy, number of bases within stems, loops or other structures, . . . ; hairpin thermodynamics of the target molecules, such as hairpin melting temperature, or Gibbs Free energy, etc; and the complexity of a sequence.

Hairpin thermodynamics of the target molecules can be much more difficult to calculate than hairpin thermodynamics of the probe, as the targets are usually much longer than the probes, Also, the boundaries of the targets are only known for targets that are well defined often by restriction digest of the end points. There are many factors the effect the target, such as the methods of labeling often generate labeled targets much shorter than the template, (especially when they are random primed, rather than end-labeled). Also enzymes used for labeling are often inefficient for labeled nucleotides and fall of the template. Additionally, there are many forms of degradation of the targets associated with its storage (e.g. formalin-Fixed paraffin-embedded DNA), or it's purification, amplification or processing). These may include, random shearing or biased shearing of the DNA.

The Complexity of a sequence can take many forms. “Complexity” is defined here as the number of bases (of the probe) that are contained within short simple repeats, such as homopolymers, dimers, trimers (e.g. ACGACGACGACG . . . ), tetramers, . . . In our current calculation of complexity, we typically consider repeats of as many as 6-nucleotides (hexamers), but there is no reason that one cannot include more.

Another set of in silico metrics relates to the homology of a probe, such as the homology score, HomLogS2B (which is described in detail in application Ser. No. 10/996,323 filed Nov. 23, 2004 and titled “Probe Design Methods and Microarrays for Comparative Hybridization and Location Analysis”, which is hereby incorporated herein, in its entirety, by reference thereto), distance to the nearest hit (not including the first specific target sequence) within the genome (or transcriptome for expression), and other scores that combine homology with the thermodynamic characteristics of the near hits. Another set of in silico metrics relates to measurable quantities that are indicative of probe performance, such as those that can be extracted from “simple” non-differential model systems, such as self-self or the male-female model systems as applied to probe selection for autosomes for CGH applications. These include the various signal measurements for the probes, the dye-biases, the cross-hybridization to targets whose copy numbers are varied in the model system (for CGH applications), differential sensitivity measurements by temperature, salt etc.

Another score related to homology is referred to as the “predicted homology response”, denoted by Sₕₒₘ. This score is similar to HomLogS2B, but instead of predicting the Signal-to-background, this score predicts the slope response of a probe based on Homology calculations alone under the assumption that the thermodynamic and other properties of the probe are ideal. This predicted homology slope can be defined as:

$${S}_{Hom} ≡ \frac{{\sum }_{j = 1}^{TargetSeq .} P ( {mm}_{j} )}{{\sum }_{i = 1}^{Genome} P ( {mm}_{i} )} \qquad ( 1 )$$

where P(mmⱼ) is a penalty term representing the signal contribution (under the specified hybridization conditions) for the hybridization of the probe of interest to each sufficiently complementary mismatch sequence within a specified target sequence, set of target sequences, or genome. The summation in the denominator is over all the sequences in the genome, or within the complex set of sequences expected to be in a sample or set of samples. The numerator represents the target sequence of interest. In the most specific case, the target sequence refers to the small specific sequence for which the probe was designed within a particular locus within a narrow region of the specific chromosome for which it was designed. In this case, the expression above can be simplified to

$${S}_{Hom} = \frac{1}{{\sum }_{i = 1}^{Genome} P ( {mm}_{i} )} \qquad ( 2 )$$

The function P(mmⱼ) can be calculated using a model for the hybridization between oligo sequences for using nearest neighbor models. This term is dependent on the number of mismatches, the distributions of mismatches through the aligned sequences, the specific mismatched bases, and the length of the overlap. In principle all possible sequences within the target sequences (or whole genome) should be considered, but in practice, only those sequences that are close (homologous enough) to the probe sequence need be considered. In the case of 60-mers probes, considering all subsequences in the genome that align with fewer than about 20 bases appears to be a sufficient approximation, yet one that still takes considerable computational resources to calculate.

In a further simplified model where we find the distances (or numbers of mismatches) between the probe and the nearest hits in the genome, the homology slope response can be approximated as

$${S}_{Hom} \approx \frac{{\sum }_{d = 0}^{D} {P}_{d} {M}_{d}}{{\sum }_{d = 0}^{D} {P}_{d} {N}_{d}} \qquad ( 3 )$$

where N_{d} represents the total number of hits at a distance d, where d is defined as the number of single-base differences between the probe of interest and the complex set of sequences, or the whole genome, and D is the maximum distance that needs to be considered. The denominator again represents the signal contributions of all probes in the complex set of sequences (including the target sequence). In Equation (3), the numerator represents either the target for the probe sequence itself, or in the case of a model system, it may represent the region of the model system's sequence that is being varied. For example, if the model system for a whole chromosome M_{d} represents the number of all hits within that chromosome at a distance d from the probe of interest, then P_{d} is the signal penalty for each target mismatch at a distance d. In this case a perfect match has P_{d}=1, and the value of P_{d} decreases as the number of mismatches increases, and as they become more destabilizing. This is an approximation because the precise penalty should be related to the exact sequences of both the target and probe sequence and related to the distributions of those mismatches, insertions and deletions. Again, the use of a nearest neighbor model within the homology search calculator can improve the accuracy of this approximation. The approximation is based on the assumption that the average signal reduction across a large number of probe-target mismatches is a good representation for any given mismatch of the same order. In the simplest approximation we can take assign a constant penalty P for each mismatched base, or base-insertion or base-deletion. In this case, we can relate a overall single-base penalty to the distance by

P_{d}≈P^{d} (4)

Still there are other homology scores, such as, maxTemp, that combine homology with the thermodynamic characteristics of the near hits. In this case, maxTemp is defined as the duplex melting temperature between the probe and the longest contiguous match within each homologous sequence in the background genome. The duplex melting temperature may be calculated by the simple formula where each matching GC-pair gets a value of two, and each matching A-T pair gets a value of 1, and the sum of these roughly approximates the melting temperature. Although this is an overly simple calculation of the melting temperature, it is used for the purpose of speed since the calculation needs to be done of all near hits in the genome.

MMClosestDuplexTm is the melting temperature of the closest mismatch to the probe sequence in the genome as calculated using a nearest neighbor model.

Model systems for CGH applications may be used that include regions of known copy number changes to establish the relationships between calculable or measurable metrics and the probe performance that can be measured in these systems. This can be accomplished by tuning parameters that characterize the performance for each of the metrics.

The X-chromosome provides a useful model system for doing this performance characterization. However, like many of the possible model systems, the X-chromosome is less than idyllic in that each probe within it does not necessarily exist at a single locus within the variable region. Additionally, there may be a number of other homologous regions within the region systematically varied by the model system that do not exactly match the intended target of the probe sequence. This is especially true for models with large contiguous regions, such as the X-chromosome or other cell lines with aberrations in a chromosome or a segment of a chromosome.

Currently the methods for calculating the homology scores, do not discriminate between probes that have multiple exact copies within the variable region (the X-chromosome) and those that have multiple copies elsewhere in the genome. For this reason, these metrics may be modified by removing the X-chromosome from the background genome set and replacing it with a string of bases consisting of the concatenated set of X-chromosome probes that are being evaluated.

When one item is indicated as being “remote” from another, this is referenced that the two items are not at the same physical location, e.g., the items are at least in different buildings, and may be at least one mile, ten miles, or at least one hundred miles apart.

“Communicating” information references transmitting the data representing that information as electrical signals over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

A “processor” references any hardware and/or software combination which will perform the functions required of it. For example, any processor herein may be a programmable digital microprocessor such as available in the form of a mainframe, server, or personal computer. Where the processor is programmable, suitable programming can be communicated from a remote location to the processor, or previously saved in a computer program product. For example, a magnetic or optical disk may carry the programming, and can be read by a suitable disk reader communicating with each processor at its corresponding station.

Reference to a singular item, includes the possibility that there are plural of the same items present.

“May” means optionally.

Methods recited herein may be carried out in any order of the recited events which is logically possible, as well as the recited order of events.

All patents and other references cited in this application, are incorporated into this application by reference except insofar as they may conflict with those of the present application (in which case the present application prevails).

### Methods, Systems and Computer Readable Media

The methods of the present invention described herein may be carried out to empirically determine probe performances and select high performing probes (e.g., probes that produce a relatively high signal from binding with an intended target and exhibit relatively low cross-hybridization) for a population of probes that are empirically tested. The population of probes selected for testing may be identified by any existing methods, including those referred to in the background section above. The present techniques can even be practiced beginning with a randomly selected set of probes, as the invention can identify probes having signal dominated by weakly bound, labeled target sequences. However, this approach is not the most efficient, since the design of an array requires probes that span the space of all genes (for gene expression experiments) that are expected to be present in an experiment that the array is designed for, or that span all locations (e.g., on a chromosome) that are to be considered during experimentation.. Accordingly, an initial set of probes to be processed by techniques described herein may be selected by best knowledge that is available to the designer, which may include bioinformatics metrics, clustering techniques, and databases that store data characterizing probes that are being considered as potential candidates for the initial set. Thus, the present techniques may be practiced in combination with one or more iterations of experimentally or computationally selected probes, or may be practiced independently on a population of existing probes or a population of probes selected by any other technique, including random selection. Although in silico metrics may help to predict probe performance, they suffer the disadvantages described above in the background section. Further, the present methods exhibit greater sensitivity for identifying probe performance and add an independent dimension to probe selection. This may also reduce or eliminate the need to iteratively test probe sets in the manners described in the background section. Further, the current methods may eliminate the need for model systems used for such iterations and may be used to select a better set of probes for use in designing arrays for gene expression, CGH or location analysis, for example.

For two identical sets of probes where one set is hybridized to a sample at a first hybridization stringency and the second set is hybridized to the identical sample at a second hybridization stringency higher than the first hybridization stringency (and where both the hybridization stringencies are within a practically effective range), the probes hybridized at the lower hybridization stringency will generally exhibit higher signals when scanned than the probes hybridized at the higher hybridization stringency. Hybridization time is typically set so that adequate signal (i.e., sufficient bonding of target to each probe) is achieved by all probes. However, specific probes (i.e., those that exhibit relatively low cross-hybridization (non-specific binding)) of the higher hybridization stringency set exhibit significantly less signal loss relative to the same probes in the lower hybridization stringency set than the signal loss exhibited by non-specific probes (i.e., those probes that exhibit relatively high amounts of cross-hybridization (non-specific binding)). That is, the stringency-sensitivity of the intensity of signals received from non-specific binding to probes is higher than the stringency-sensitivity to hybridization of the intensity of signals received from specific binding to probes.

Assuming that effects on hybridization such as diffusion are minor (e.g., hybridization protocol times may be in the neighborhood of about 12 to 45 hours to ensure adequate diffusion to all probes, although use of a microwave heat source that applies traveling microwave waves to an array may provide more uniform radiative heat to more accurately and efficiently deliver heat energy, create convective circulation, and thereby decrease the hybridization time required), the population of bound sequence fragments from a target solution applied to a probe can be described by (e.g., see Dai et al., “Use of hybridization kinetics for differentiating specific from non-specific binding to oligonucleotide microarrays”, Nucleic Acids Research, 2002, Vol. 30 No. 16, 2002 Oxford University Press, which is hereby incorporated herein, in its entirety, by reference thereto).

$$\begin{matrix}I ( t , T ) = \frac{OL}{K + \frac{O}{{N}_{O} V} ( 1 - {e}^{- t / τ} )} & ( 5 ) \\ K = {e}^{Δ G / RT} & ( 6 ) \\ τ = {{k}_{f}^{- 1} ( K + \frac{O}{{N}_{O} V} )}^{- 1} & ( 7 )\end{matrix}$$

where:
I=the population or number of bound sequences on a probe from the biological sample. The signal extracted from a probe is monotonically proportional with I;
t=time, in seconds;
T=absolute temperature, in Kelvin;
O=the number of sequence fragments (e.g., nucleotide sequences; oligomers) bound to the probe;
L=the target concentration in moles/liter of the target solution;
K=the kinetic equilibrium disassociation constant, in moles/liter;
N_{O}=Avogadro's number;
V=the volume of hybridization solution, in liters;
τ=a characteristic time over which equilibrium of the hybridization is achieved;
k_{f}=a kinetic parameter as defined in Dai et al, cited above, which denotes the forward time rate of the hybridization process; and
ΔG =the free-energy difference, in kilocalories, fro probe binding at 37C. ΔG changes modestly over the practical ranges of application and therefore is considered as a constant of T, e.g., see SantaLucia, Jr., “A unified view of polymer, dumbbell, and oligonucleotide DNA nearest-neighbor thermodynamics”, Proc. Natl. Acad. Sci. USA, Vol 95, pp. 1460-1465, February 1998, Biochemistry, which is hereby incorporated herein, in its entirety, by reference thereto.

The rate of change of I with respect to T is described by:

$$\begin{matrix}r ( t , T ) = \frac{\partial I}{\partial T} = - [ \frac{OL}{{( K + \frac{O}{{N}_{O} V} )}^{2}} ( 1 - {e}^{- t / τ} ) - \frac{OL}{K + \frac{O}{{N}_{O} V}} {e}^{- t / τ} {tk}_{f} ] \frac{\partial K}{\partial T} & ( 8 ) \\ where \frac{\partial K}{\partial T} = - \frac{Δ G}{{RT}^{2}} {e}^{Δ G / RT} & ( 9 )\end{matrix}$$

As noted, ΔG is considered as a constant of T. For example K is typically ˜10⁻¹⁰ for ˜25-mer oligomers having ΔG of about −14 kcal at 37C. Oligomers having 60-mers create a much greater drop in G, e.g., see Zhang et al., “Competitive Hybridization Kinetics Reveals Unexpected Behavior Patterns”, Biophys J BioFAST, Aug. 26, 2005, doi:10.1529/biophysj.104.058,552, which is hereby incorporated herein, in its entirety, by reference thereto. Also, typically

$$\frac{O}{{N}_{0} V}$$

is around 10⁻⁶ moles/liter.

### Therefore,

$$K \text{<<} \frac{O}{{N}_{0} V} , and K + \frac{O}{{N}_{0} V}$$

is essentially

$$\frac{O}{{N}_{0} V} .$$

At equilibrium (i.e., t≧τ) the rate equation (i.e., equation (4)) becomes:

$$r ( ∞ , T ) = [ \frac{OL}{{( K + \frac{O}{{N}_{O} V} )}^{2}} ] \frac{Δ G}{{RT}^{2}} {e}^{Δ G / RT} \qquad ( 10 )$$

Considering an example for 60-mer probes, a typical “noise sequence” (i.e., a sequence for which a probe has not been designed to specifically bind with, and thus will only bind to probes by non-specific binding (i.e., cross-hybridization)) in a target solution may have a ΔG (which we designate as ΔGₙ here) of ˜−30 kcal and having a stringency rate designated here by rₙ. A typical “specific sequence” (i.e., a sequence for which a probe exists that this sequence will specifically bind to, i.e., the probe has a complementary sequence to the specific sequence) may have a ΔG (which we designate as ΔGₛ here) of ˜−80 kcal and a stringency rate designated here by rₛ. Given these exemplary ΔG values, a relative stringency rate, rₙ/rₛ at equilibrium can be defined as follows:

$$\frac{{r}_{n} ( ∞ , T )}{{r}_{s} ( ∞ , T )} = \frac{Δ {G}_{n} {e}^{Δ {G}_{n} / RT}}{Δ {G}_{s} {e}^{Δ {G}_{s} / RT}} \geq {e}^{50} \qquad ( 11 )$$

Hence, a decrease in the population of noise sequences on a probe will be much greater than a decrease in the population of the specific sequence for that probe as the hybridization temperature is increased during hybridization processing. This relationship is also true for the non-equilibrium kinetics, e.g., over the course of the hybridization process before it reaches equilibrium. That is, the stringency rate for specific sequences is much greater than that for noise sequences for all time t>0.

In addition to hybridization temperatures, chemical process steps can also impact the stringency rates of specific and noise sequences. For example, that additions of salts and/or formamide to the hybridization solution may alter the stringency rates. In general, the stringency rate of a specific sequence relative to the stringency rate of non-specific (noise) sequences will exhibit a major difference, as described above, regardless of the source(s) driving the stringency for each probe.

Since, in general, noise signals (e.g., signals from non-specific bindings) tend to be proportional to true signal (i.e., signal from specific binding to a probe), an appropriate normalization of intensity signals for calculation of relative stringency rates should be calculated by either converting to a relative decrease (i.e., divide the signal decrease between the same probe at the two different hybridization stringencies by the signal intensity from the probe processed at the higher hybridization stringency), or by calculation the log of the intensity signals for stringency rate calculations, e.g., delta=LogI_{T=60}−LogI_{T=70}. The logarithmic transform (Log transform) inherently provides correct statistical weighting for intensity.

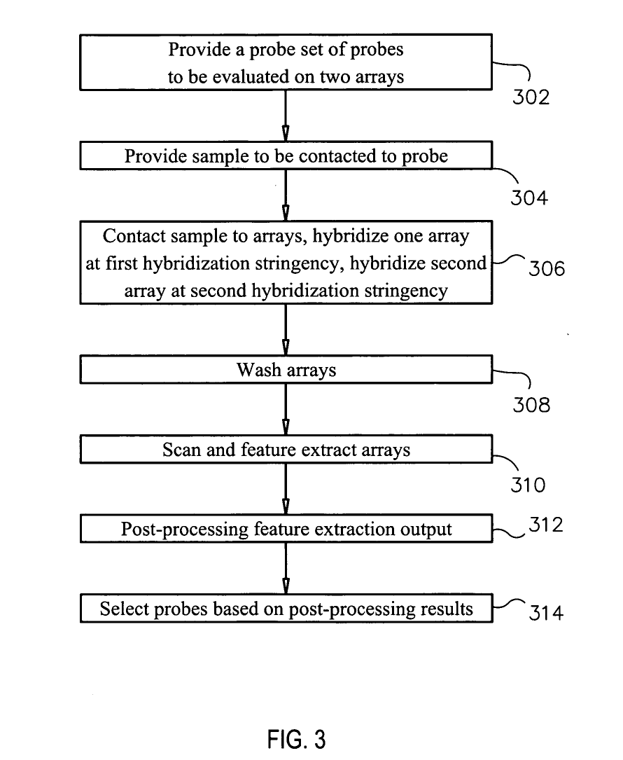

In view of the above, probe selection for array design can be expedited, as well as improved in specificity by analyzing probes that are hybridized at different hybridization stringencies. FIG. 3 illustrates events that may be carried out to estimate probe performance for selection of probes exhibiting the best performance for an array design. At event 302 a set of probes to be evaluated is provided, in order to select a subset of the best performing probes for placement on an array. The set of probes to be evaluated may be selected according to any of the techniques described in the background section, or any other existing technique for probe selection currently used for array design, or even randomly.

The set of probes is provided on two arrays, wherein each array includes the same probes to be compared against one another, so that signals extracted from probes having been processed on the first array can be compared with signals extracted from the same probes having been processed (although under different hybridization stringency conditions) on the second array.

At event 304, a sample is provided that is to be contacted to the probes on the arrays to hybridize sequences in the sample to probes on the array. Typically the set of probes will contain probes that are designed to specifically bind to specific sequences in the sample. The sample contacted to the first array should be the same as the sample contacted to the second array. Accordingly, a single sample is typically provided is divided in two for application to the two arrays. For example, for two-channel scanning, a sample A may be labeled with Cy-3 (cyanine-3) green fluorescent dye and a sample B may be labeled with Cy-5 (cyanine-5) red fluorescent dye. Samples A and B are then combined to form sample AB and mixed for random distribution of the samples A and B in sample AB. Sample AB is then divided into two aliquots of sample AB.

At event 306, sample AB is next contacted to the first array (by contact with the first aliquot) and the second array (by contact with the second aliquot) and the first array is hybridized at a first hybridization stringency, while the second array is hybridized at a second hybridization stringency that is different than the first hybridization stringency. As one non-limiting example, the first hybridization stringency may include a hybridization temperature of 60° C. and the second hybridization stringency may include a hybridization temperature of 70° C.

After the hybridization event 306, wherein hybridization may be carried out until equilibrium is reached, or hybridization time may be set so that adequate signal (i.e., sufficient bonding of target to each probe) is achieved by all probes, both arrays may be washed at event 308, according to existing wash techniques to remove unbound sequences from the probes.

Next the arrays may be scanned and feature extracted at event 310 to obtain feature extraction outputs including intensity signals from the probes that are characteristic of the sequences (and amounts thereof) that have bound to the probes, as indicated by the luminescence of the fluorescing dyes as they are illuminated during the scanning process, as is known.

At event 312, the signal intensity data outputted as a result of the feature extraction of the array is post-processed to remove the scanner offset for both the red and green channel signals. These signals are then converted to natural log values by taking the natural logarithm of the signals from which the scanner offset values were removed. The natural log values of the green channel signals are referred to here as gLnNMS, wherein “NMS” refers to “net mean signal”, which is the signal minus a scanner offset value, as is commonly known, and the natural log values of the red channel signals are referred to as rLnNMS. The mean of the log values of the red/green log ratio signals may be calculated to average out dye differential issues that may be result between the red and green dyes. The mean red/green dye natural log signal value is referred to here as rgLnNMS. For each probe existing on both arrays that is to be compared, the relative change in signal between post-processed signal from the probe on the first array and the post-processed signal from the same probe on the second array is calculated. Such a calculated signal difference is referred to as rgLnNMS6070, for signals extracted from array that were hybridized at 60° C. and 70° C., respectively, for example.

The signal difference values (i.e., stringency difference metrics) for each probe may then be compared to select those probes that indicate the best, or better performance than other probes considered. Probes exhibiting the relatively lower signal difference values are chosen as the better performing probes. The probes that show relatively higher signal difference values indicate that a larger proportion of the sequences bound to the probe at the lower hybridization stringency were non-specifically bound (noise) sequences, since noise sequences have a faster stringency rate than specific sequences, as described above. Thus, probes from which a relatively high difference in signal between the probe processed at a lower hybridization stringency and the probe processed at a relatively higher hybridization stringency should be avoided, since these probes have bound with a relatively higher percentage of non-specific sequences than those probes from which a relatively low difference in signal was calculated. These stringency difference metrics may be used in combination with other defined metrics to create an ensemble score for selection of best performing probes, or, used separately that can be used to identify the worst performing probes for elimination from possible selection for an array design. Thus a combination of metrics (which may include scores calculated in silico) may be used to select probes, or stringency metrics may be used as an individual indicator of the better performing probes. When ensemble scores are used, the population of ensemble scores having been calculated can be sorted and plotted as a sigmoid chart to identify the extreme, worst-performing probes, which can then be replaced by new candidate probes and processing as described above can be iterated with the new set of probes. Alternatively, a predetermined percentage of the worst performing probes defined by the sigmoid plot can be replaced with new probe candidates and the processing can be iterated with the new set of probes.

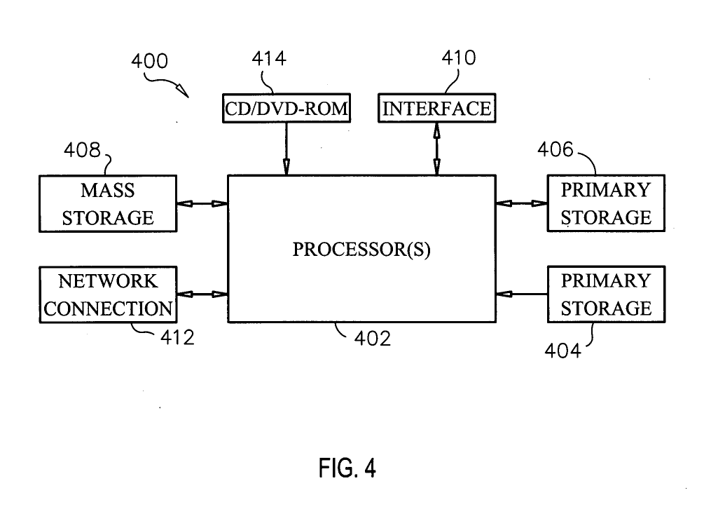

FIG. 4 is a schematic illustration of a typical computer system that may be used to perform procedures described above. The computer system 400 includes any number of processors 402 (also referred to as central processing units, or CPUs) that are coupled to storage devices including primary storage 406 (typically a random access memory, or RAM), primary storage 404 (typically a read only memory, or ROM). As is well known in the art, primary storage 404 acts to transfer data and instructions uni-directionally to the CPU and primary storage 406 is used typically to transfer data and instructions in a bi-directional manner Both of these primary storage devices may include any suitable computer-readable media such as those described above. A mass storage device 408 is also coupled bi-directionally to CPU 402 and provides additional data storage capacity and may include any of the computer-readable media described above. Mass storage device 408 may be used to store programs, data and the like and is typically a secondary storage medium such as a hard disk that is slower than primary storage. It will be appreciated that the information retained within the mass storage device 408, may, in appropriate cases, be incorporated in standard fashion as part of primary storage 406 as virtual memory. A specific mass storage device such as a CD-ROM or DVD-ROM 414 may also pass data uni-directionally to the CPU.

CPU 402 is also coupled to an interface 410 that includes one or more input/output devices such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 402 optionally may be coupled to a computer or telecommunications network using a network connection as shown generally at 412. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for calculating differences in signals from a probe where a first probe was processed at a first hybridization stringency in one instance and another probe the same as the first probe was processed at a second hybridization stringency. As another example instructions may be provided to one or more CPU's 402 to perform feature extraction from an electronic image of an array having been scanned. Further, instructions may be included for operating a scanner connected to computer system 400 to scan an array and output an electronic image of the scanned array. Outputs of these processes may be displayed on a user interface 410, such as a monitor and/or outputted in hard copy form such as via a printer and/or transmitted, such as by email, fax or other electronic means. Instructions for these processes may be stored on mass storage device 408 or 414, or another storage device accessible to system 400 via network connection 412, and executed on CPU 408 in conjunction with primary memory 406.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

### EXAMPLE

The following example is put forth so as to provide those of ordinary skill in the art with a complete disclosure and description of how to make and use the present invention, and is not intended to limit the scope of what the inventors regard as their invention nor are they intended to represent that the experiment below is the only experiment performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Two arrays (i.e., 1 channel-flip pair of arrays) were hybridized at 60° C. and two arrays (another channel-flip pair of arrays the same as the first channel-flip pair of arrays) were hybridized at 70° C. The arrays used were X2A CGH zoom-in X arrays from Agilent Technologies, Inc. (Palo Alto, Calif.), each including 44,000 features, meaning that most probes were for locations on the X-chromosome. The samples used were male human genomic DNA and female human genomic DNA samples from Promega (Fitchburg, Wis.), as indicated in Table 1 below.

TABLE 1

| Product | Cat.# | Size | Qty. |
|---|---|---|---|
| Human | G1471 | 100 μg | 1 |
| Genomic DNA: | | | |
| Male | | | |
| Human | G1521 | 100 μg | 1 |
| Genomic DNA: | | | |
| Female | | | |

Greater than 90% of the DNA strands in the samples used were longer than 50 kb in size as measured by pulsed-field gel electrophoresis. The samples were stored at 4° C. prior to use in 10 mM Tris-HCl (pH 8.0), 1 mM EDTA. Because the hybridizations were performed with female/male samples, the probes specific to locations on the X-chromosome were expected to show a 2:1 ratio (female/male) which is a LogRatio value of about 0.3.

Table 2 below shows the samples that were hybridized on the channel-flip pairs of arrays. All samples were of the same composition, but labeled with different sample numbers for tracking (and with pairs labeled with different dyes), as indicated. In the first dye-flip pair of arrays, samples 01 and 02 were hybridized at 60° C. Sample 01 had the male DNA labeled with Cy-5 dye and the female sample labeled with Cy-3 dye. Sample 02 had the male DNA labeled with Cy-3 dye and the female DNA labeled with Cy-5 dye. In the second dye-flip pair of arrays, samples 07 and 08 were hybridized at 70° C. Sample 07 had the male DNA labeled with Cy-5 dye and the female DNA labeled with Cy-3 dye. Sample 08 had the male DNA labeled with Cy-3 dye and the female DNA labeled with Cy-5 dye.

TABLE 2

| Sample | Array | Barcode | Scan order |
|---|---|---|---|
| Sample 01 at 60 C M Cy-5 F Cy-3 | 2513637 | 10024 | 3 |
| Sample 02 at 60 C M Cy-3 F Cy-5 | 2513637 | 10025 | 2 |
| Sample 07 at 70 C M Cy-5 F Cy-3 | 2513637 | 10030 | 6 |
| Sample 08 at 70 C M Cy-3 F Cy-5 | 2513637 | 10031 | 7 |

Some probes, such as for example, pseudo-autosomal probes and probes that had very high melting temperatures (Tm), relative to the other probes on the arrays, were excluded from the data analysis. To exclude these probes, probe filters were applied, using Agilent Feature Extraction software, version FE 8.1 (Agilent Technologies, Inc., Palo Alto, Calif.), according to the following pseudocode:
To remove pseudo-autosomal probes between chrX and chrY and also some bad probes, use a SQL join selection as follows:
SQL Join chrX zoomin with 5 calculated metric selections:
SELECT ChrX_D1_Metrics.ProbelD,
ChrX_D1_Metrics.DuplexTm, ChrX_D1_Metrics.MaxSubSeqTm,
ChrX_D1_Metrics.Complexity, ChrX_D1_Metrics.maxTmp,
ChrX_D1_Metrics.HomLogS2B, ChrX_D1_Metrics.Score
FROM ChrX_D1_Metrics
WHERE (((ChrX_D1_Metrics.DuplexTm)<90 And
(ChrX_D1_Metrics.DuplexTm)>70) AND
((ChrX_D1_Metrics.MaxSubSeqTm)<65) AND
((ChrX_D1_Metrics.Complexity)<30) AND
((ChrX_D1_Metrics.maxTmp)<55) AND
((ChrX_D1_Metrics.HomLogS2B)>4));
The SQL join produced 33020 chrX probes.

After filtering to remove pseudo-autosomal probes and probes having a very high Tm, 33,020 probes remained for processing. The feature extraction data from the remaining probes was post-processed in a manner as described above, and according to the following pseudocode description (performed using JMP*SAS software):
Remove scanner offset from the FE-extracted signals and convert to
natural log values:
New Column(“rLnNMS”, Numeric, Continuous, Format(“Best”, 10),
Formula(Log(:rMeanSignal-:rOffsetUsed)));
New Column(“gLnNMS”, Numeric, Continuous, Format(“Best”, 10), Formula(Log(:gMeanSignal-:gOffsetUsed)));
Calculate mean dye Ln signal to average out dye differential issues:
New Column(“rgLnNMS”, Numeric, Continuous, Format(“Best”, 10), Formula(Mean(:rLnNMS, :gLnNMS)));
Select the 60C and 70C XX values from rgLnNMS to use in the method, labeled
rgLnNMS XX 60C and rgLnNMS XX 70C.
Calculate difference in these values between 60C and 70C hyb′ T for XX target:
New Column(“rgLnNMSXX6070”, Numeric, Continuous,
Format(“Best”, 10), Formula(:rgLnNMS XX 60C-:rgLnNMS XX 70C));
Calculate mean channel-flip Log ratio for 70C hyb′:
New Column(“LogRatio70”, Numeric, Continuous, Format(“Best”, 10), Formula(Mean(LogRatio XX 70C,-:LogRatio XY 70C)));
Calculate channel-flip error for 70C hyb′:
New Column(“flipError”, Numeric, Continuous, Format(“Best”, 10),
Formula(Mean(:LogRatio XY 70C, :LogRatio XX 70C)));

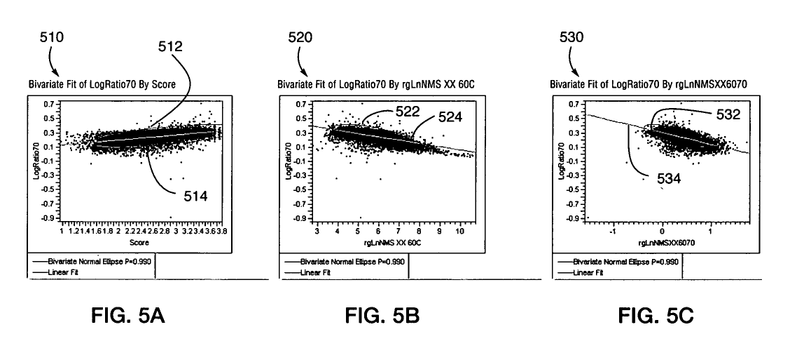

FIGS. 5A-5C show plots 510, 520 and 530 of a bivariate fit of the LogRatio 70 log ratio values versus scores for the same. “Scores” are based on known bioinformatics calculations such as melting temperature (Tₘ), which are combined to form the best bioinformatics scores to predict performance of probes. The stringency differential metrics, calculated as described herein are compared to the best bioinformatics scores (Scores) by correlation with the expected trend in signal ratio values between XX and XY sample data. A bivariate fit is made of the LogRatio 70 log ratio values against rgLnNMS XX 60C values, wherein each LogRatio 70 value is a log ratio of the net mean signal of a female sample to the net mean signal of a male sample, wherein both samples were processed on the same probe at 70° C. (i.e., two-color arrays, where no Y-chromosome probes were used) and wherein each rgLnNMSXX 60 value is the mean of the Ln net mean signal of female sample and the Ln net mean signal of a male sample, both processed at 60° C. This stringency metric evaluates the correlation of stringency-signal difference with ratio performance at 70° C. for each probe. Similarly, a bivariate fit is made of the LogRatio 70 log ratio values versus rgLnNMSXX6070 values, wherein each rgLnNMSXX6070 value is the difference between rgLnNMS XX 60 (mean of Ln net mean signal of female sample and Ln net mean signal of a male sample, both processed at 60° C.) and rgLnNMS XX 70 (mean of Ln net mean signal of female sample to and Ln net mean signal of a male sample, both processed at 70° C. A bivariate, normal ellipse (P=0.990) is plotted around the data in each of the plots as 512, 522 and 532, respectively, to show the bivariate fits. Linear fits of the data are shown by lines 514, 524 and 534, respectively.

Table 3 displays the results of an analysis of variance (ANOVA) regression analysis carried out with respect to the data plotted in FIG. 5A. The analysis was carried out using JPP*SAS software version 5.1.2.(JMP Software, Cary, N.C.).

TABLE 3
Correlation

| | | | | Signif. | |
|---|---|---|---|---|---|
| Variable | Mean | Std. Dev. | Correlation | Prob. | Number |
| Score | 2.814185 | 0.431692 | 0.608315 | 0.0000 | 33020 |
| LogRatio70 | 0.264696 | 0.052417 | | | |

Linear Fit

LogRatio70 = 0.0568338 + 0.0738624 Score

Summary of Fit

| | |
|---|---|
| RSquare | 0.370047 |
| RSquare Adj. | 0.370028 |
| Root Mean Square Error | 0.041603 |
| Mean of Response | 0.264696 |
| Observations (or Sum Wghts) | 33020 |

Parameter Estimates

| Term | Estimate | Std. Error | t Ratio | Prob > \|t\| |
|---|---|---|---|---|
| Intercept | 0.0568338 | 0.00151 | 37.64 | <.0001 |
| Score | 0.0738624 | 0.00053 | 139.27 | 0.0000 |

Table 4 displays the results of an analysis of variance regression (ANOVA) analysis carried out with respect to the data in FIG. 5B.

TABLE 4
Correlation

| | | | | Signif. | |
|---|---|---|---|---|---|
| Variable | Mean | Std. Dev. | Correlation | Prob. | Number |
| rgLnNMSXX | 5.715032 | 0.699989 | −0.62238 | 0.0000 | 33020 |
| 60 C. | | | | | |
| LogRatio70 | 0.264696 | 0.052417 | | | |

Linear Fit

LogRatio70 = 0.5310822 + 0.0466114 rgLnNMSXX 60 C.

Summary of Fit

| | |
|---|---|
| RSquare | 0.387353 |
| RSquare Adj. | 0.387334 |
| Root Mean Square Error | 0.041028 |
| Mean of Response | 0.264696 |
| Observations (or Sum Wghts) | 33020 |

Parameter Estimates

| Term | Estimate | Std. Error | t Ratio | Prob > \|t\| |
|---|---|---|---|---|
| Intercept | 0.5310822 | 0.001857 | 285.92 | 0.0000 |
| rgLnNMSXX 60 C. | −0.046611 | 0.000323 | −144.5 | 0.0000 |

Table 5 displays the results of an analysis of variance (ANOVA) regression nalysis carried out with respect to the data in FIG. 5C.

TABLE 5
Correlation

| | | | Cor- | Signif. | |
|---|---|---|---|---|---|
| Variable | Mean | Std. Dev. | relation | Prob. | Number |
| rgLnNMSXX6070 | 0.272904 | 0.217698 | −0.67777 | 0.0000 | 33020 |
| LogRatio70 | 0.264696 | 0.052417 | | | |

Linear Fit

LogRatio70 = 0.3092321 − 0.1631917 rgLnNMSXX6070

Summary of Fit

| | |
|---|---|
| RSquare | 0.459375 |
| RSquare Adj. | 0.459359 |
| Root Mean Square Error | 0.038541 |
| Mean of Response | 0.264696 |
| Observations (or Sum Wghts) | 33020 |

Parameter Estimates

| Term | Estimate | Std. Error | t Ratio | Prob > \|t\| |
|---|---|---|---|---|
| Intercept | 0.3092321 | 0.00034 | 909.19 | 0.0000 |
| rgLnNMSXX6070 | −0.163192 | 0.000974 | −167.5 | 0.0000 |

By comparing the outputs in the above Tables 3-5, it can be observed that the correlation to LogRatio70 (67.8%) between the probe differences in signals between 60° C. and 70° C. (i.e., rgLnNMS6070) and the LogRatio values at 70° C. (i.e., LogRatio70) is higher than the correlation to LogRatio70 (62.2%) between the probe signal values at 60° C. (i.e., rgLnNMS XX 60) and the LogRatio values at 70° C. (i.e., LogRatio70), and is also higher than the correlation to LogRatio70 (60.8%) between the in silico metrics calculated for probe performance (i.e., Score) and the LogRatio values at 70° C (i.e., LogRatio70). The stringency difference metric has better correlation (sensitivity) to probe performance at 70° C. than the other two metrics, which would heretofore have been considered “best metrics”. Since the correlation is negative in sign, a smaller difference metric value indicates better signal ratio values, which are near Log₂=0.3, as expected, and as shown in the plots.

Tables 6-9 show the results of three more ANOVA analyses carried out on the data in this example. In Table 6, correlations of combination of three classes of probe properties were explored: signal intensity of probes at 60° C., calculated metrics of the probes (i.e., Score), and hybridization stringency (in this case, temperature) impact on signal intensity from 60° C. to 70° C. The correlation of the best combination of these three classes is listed in the Effect Tests below, and was calculated to be 77%.

TABLE 6
Response LogRatio70
Summary of Fit

| | |
|---|---|
| RSquare | 0.595714 |
| RSquare Adj. | 0.595641 |
| Root Mean Square Error | 0.033331 |
| Mean of Response | 0.264696 |
| Observations (or Sum Wghts) | 33020 |

Effect Tests

| | | | Sum of | | |
|---|---|---|---|---|---|
| Source | Nparam | DF | Squares | F Ratio | Prob > F |
| rgLnNMSXX 60 C. | 1 | 1 | 0.4819372 | 433.7951 | <.0001 |
| rgLnNMSXX 60 C. * | 1 | 1 | 2.1868492 | 1968.398 | 0.0000 |
| rgLnNMSXX 60 C. | | | | | |
| rgLnNMSXX6070 | 1 | 1 | 2.2363776 | 2093.989 | 0.0000 |
| rgLnNMSXX6070 * | 1 | 1 | 0.9618897 | 865.8037 | <.0001 |
| rgLnNMSXX6070 | | | | | |
| Score | 1 | 1 | 1.9004452 | 1710.604 | 0.0000 |
| Score * Score | 1 | 1 | 0.1144353 | 103.0040 | <.0001 |

By reviewing the F Ratio values, it can be observed that the hybridization stringency impact on intensity from 60° C. to 70° C. (i.e., rgLnNMSXX6070, with an F Ratio score of 2093.989) had the most significant impact on probe quality as the p-value (i.e., Prob>F value) is <0.05. Since all of the Prob>F values shown are less than 0.05, all values are significant, albeit at varying levels of significance.

In reviewing the interaction significance p-values (i.e., Prob>F) (not shown), the interactions between the three classes were indicated to not be important, i.e., no p-values were less than 0.05. The quadratics terms (i.e., rgLnNMS XX 60C*rgLnNMS XX 60C, rgLnNMSXX607*rgLnNMSXX6070 and Score*Score) are important, as the Prob>F values are less than 0.005, indicating that these values show nonlinear relationships.

It is noted that the parameters for which scores and calculations were generated for the above tables are standard parameters calculated when performing ANOVA analysis, for example using available software products such as JMP*SAS or Rosetta ROC, for example. For further detailed discussion of ANOVA analysis, see application Ser. No. 11/026,484 filed Dec. 30, 2004 and titled “Methods and Systems for Fast Least Squares Optimization for Analysis of Variance with Covariants” and application Ser. No. 11/198,362 filed Aug. 4, 2005 and titled “Metrics for Characterizing Chemical Arrays Based on Analysis of Variance (ANOVA) Factors”, both of which are hereby incorporated herein, in their entireties, by reference thereto.

Table 7 shows the results obtained after removing the property representing the hybridization stringency impact on signal intensity from 60° C. to 70° C. (rgLnNMSXX6070), so that only two classes of probe properties were explored: signal intensity of probes at 60° C. and calculated metrics of the probes (i.e., Score). This analysis showed that the signal intensity at 60° C. was the most important factor, as indicated by its combined F ratio score (linear plus quadratic F-scores). This best least squares regression multivariate combination of metrics correlation to LogRatio70 was calculated to be 73%.

TABLE 7
Response LogRatio70
Summary of Fit

| | |
|---|---|
| RSquare | 0.537207 |
| RSquare Adj. | 0.537151 |
| Root Mean Square Error | 0.035661 |
| Mean of Response | 0.264696 |
| Observations (or Sum Wghts) | 33020 |

Effect Tests

| | | | Sum of | | |
|---|---|---|---|---|---|
| Source | Nparam | DF | Squares | F Ratio | Prob > F |
| rgLnNMSXX 60 C. | 1 | 1 | 3.0327614 | 2384.847 | 0.0000 |
| rgLnNMSXX 60 C. * | 1 | 1 | 5.5781303 | 4386.428 | 0.0000 |
| rgLnNMSXX 60 C. | | | | | |
| Score | 1 | 1 | 3.5336932 | 2778.761 | 0.0000 |
| Score * Score | 1 | 1 | 0.2988926 | 235.0377 | <.0001 |

In Table 8, correlations of combination of four classes of probe properties were explored: (1) signal intensity of probes at 60° C., (2) calculated metrics of the probes (i.e., metric used to calculate Score), (3) hybridization stringency (in this case, hybridization temperature) impact on signal intensity from 60° C. to 70° C. (i.e., rgLnNMSXX6070), and (4) channel flip error. Channel flip error is the average of each probe signal ratio over two arrays that make up a flipped pair, where the two samples are labeled as red and green in the first array of the pair, and (flipped) as green and red, respectively, in the second array of the pair. Given the symmetry, the calculated average should be zero, but it typically is not zero due to sample-specific dye bias and array-array variations. Hence the average becomes an error metric (channel flip error) that is indicative of such bias and random variations. The correlation of the regression multivariate model to the 70° C. signal ratios across all XX probes was calculated to be 80%.

TABLE 8
Response LogRatio70
Summary of Fit

| | |
|---|---|
| RSquare | 0.642905 |
| RSquare Adj. | 0.642602 |
| Root Mean Square Error | 0.031336 |
| Mean of Response | 0.264696 |
| Observations (or Sum Wghts) | 33020 |

Analysis of Variance

| Source | DF | Sum of Squares | Mean Square | F Ratio |
|---|---|---|---|---|
| Model | 28 | 58.324250 | 2.08301 | 2121.294 |
| Error | 32991 | 32.395583 | 0.00098 | Prob > F |
| C. Total | 33019 | 90.719833 | | 0.0000 |

Effect Tests

| | | | Sum of | | |
|---|---|---|---|---|---|
| Source | Nparam | DF | Squares | F Ratio | Prob > F |
| rgLnNMSXX 60 C. | 1 | 1 | 0.6038897 | 614.9889 | <.0001 |
| rgLnNMSXX6070 | 1 | 1 | 2.5474033 | 2594.224 | 0.0000 |
| flipError | 1 | 1 | 0.0996677 | 101.4996 | <.0001 |
| Duplex Tm | 1 | 1 | 0.0066025 | 6.7239 | 0.0095 |
| MaxSubSeqTm | 1 | 1 | 0.7087082 | 721.7339 | <.0001 |
| Complexity | 1 | 1 | 0.0093273 | 9.4988 | 0.0021 |
| maxTmp | 1 | 1 | 0.0968421 | 98.6220 | <.0001 |
| HomLogS2B | 1 | 1 | 0.0075697 | 7.7088 | 0.0055 |
| rgLnNMSXX 60 C. * | 1 | 1 | 2.6511836 | 2699.911 | 0.0000 |
| rgLnNMSXX 60 C. | | | | | |
| rgLnNMSXX6070 * | 1 | 1 | 0.0555194 | 56.5398 | <.0001 |
| rgLnNMSXX6070 | | | | | |
| rgLnNMSXX 60 C. * | 1 | 1 | 0.0546337 | 55.6379 | <.0001 |
| flipError | | | | | |
| flipError * flipError | 1 | 1 | 1.6078101 | 1637.361 | 0.0000 |
| rgLnNMSXX 60 C. * | 1 | 1 | 0.3969948 | 404.2914 | <.0001 |
| DuplexTm | | | | | |
| flipError * DuplexTm | 1 | 1 | 0.0824427 | 83.9580 | <.0001 |
| DuplexTm * | 1 | 1 | 0.0835403 | 85.0757 | <.0001 |
| DuplexTm | | | | | |
| rgLnNMSXX6070 * | 1 | 1 | 0.2627623 | 267.5918 | <.0001 |
| MaxSubSeqTm | | | | | |
| MaxSubSeqTm * | 1 | 1 | 0.0352447 | 35.8924 | <.0001 |
| MaxSubSeqTm | | | | | |
| rgLnNMSXX 60 C. * | 1 | 1 | 0.2490512 | 253.6286 | <.0001 |
| maxTmp | | | | | |
| flipError * maxTmp | 1 | 1 | 0.0414989 | 42.2616 | <.0001 |
| DuplexTm * maxTmp | 1 | 1 | 0.0183987 | 18.7369 | <.0001 |
| MaxSubSeqTm * | 1 | 1 | 0.0756397 | 77.0299 | <.0001 |
| maxTmp | | | | | |
| maxTmp * maxTmp | 1 | 1 | 0.0881677 | 89.7882 | <.0001 |
| rgLnNMSXX 60 C. * | 1 | 1 | 0.0322199 | 32.8121 | <.0001 |
| HomLogS2B | | | | | |
| rgLnNMSXX6070 * | 1 | 1 | 0.0565968 | 57.6370 | <.0001 |
| HomLogS2B | | | | | |
| flipError * | 1 | 1 | 0.0635314 | 64.6991 | <.0001 |
| HomLogS2B | | | | | |
| DuplexTm * | 1 | 1 | 0.0185186 | 18.8590 | <.0001 |
| HomLogS2B | | | | | |
| maxTmp * | 1 | 1 | 0.0055400 | 5.6418 | 0.0175 |
| HomLogS2B | | | | | |
| HomLogS2B * | 1 | 1 | 0.0066391 | 6.7612 | 0.0093 |
| HomLogS2B | | | | | |

In reviewing the output values in Table 8, it can be observed that some interactions between classes and quadratics of classes (same source times itself) were important contributors to the correlation. Since all effects were significant as indicated by their very low p-values (i.e., Prob>F scores), the F-Ratio scores were used as a quantitative relative measure of importance among these significant effects. The F Ratio scores show that the hybridization stringency impact on intensity from 60° C. to 70° C. (i.e., rgLnNMSXX6070, with an F Ratio score of 2594.224) had the most important impact on probe quality of any single source.

Table 9 shows the results of a correlation study done that included all of the classes of sources analyzed in Table 8, except for the hybridization stringency (hybridization temperature) impact on signal intensity from 60° C. to 70° C. (i.e., rgLnNMSXX6070). The correlation of the multivariate regression model to LogRatio70 was calculated to be 77%, which is less that the 80% achieved with rgLnNMSXX6070 included in the model described above with regard to Table 8.

TABLE 9
Response LogRatio70
Summary of Fit

| | |
|---|---|
| RSquare | 0.592078 |
| RSquare Adj. | 0.591806 |
| Root Mean Square Error | 0.033489 |
| Mean of Response | 0.264696 |
| Observations (or Sum Wghts) | 33020 |

Analysis of Variance

| Source | DF | Sum of Squares | Mean Square | F Ratio |
|---|---|---|---|---|
| Model | 22 | 53.713230 | 2.44151 | 2176.977 |
| Error | 32997 | 37.006602 | 0.00112 | Prob > F |
| C. Total | 33019 | 90.719833 | | 0.0000 |

Effect Tests

| | | | Sum of | | |
|---|---|---|---|---|---|
| Source | Nparam | DF | Squares | F Ratio | Prob > F |
| rgLnNMSXX 60 C. | 1 | 1 | 3.3292009 | 2968.488 | 0.0000 |
| flipError | 1 | 1 | 0.3263963 | 291.0318 | <.0001 |
| Duplex Tm | 1 | 1 | 0.0090565 | 8.0752 | 0.0045 |
| MaxSubSeqTm | 1 | 1 | 1.3432719 | 1197.731 | <.0001 |
| Complexity | 1 | 1 | 0.0456719 | 40.7234 | <.0001 |
| maxTmp | 1 | 1 | 0.3461367 | 308.6334 | <.0001 |
| HomLogS2B | 1 | 1 | 0.1890188 | 188.5389 | <.0001 |
| rgLnNMSXX 60 C. * | 1 | 1 | 3.2595603 | 2906.393 | 0.0000 |
| rgLnNMSXX 60 C. | | | | | |
| rgLnNMSXX 60 C. * | 1 | 1 | 0.0187395 | 16.7091 | <.0001 |
| flipError | | | | | |
| flipError * flipError | 1 | 1 | 1.9288066 | 1719.824 | 0.0000 |
| rgLnNMSXX 60 C. * | 1 | 1 | 0.1953200 | 174.1574 | <.0001 |
| DuplexTm | | | | | |
| flipError * DuplexTm | 1 | 1 | 0.0553761 | 49.3762 | <.0001 |
| DuplexTm * | 1 | 1 | 0.1306381 | 116.4837 | <.0001 |
| DuplexTm | | | | | |
| MaxSubSeqTm * | 1 | 1 | 0.1888504 | 168.3888 | <.0001 |
| MaxSubSeqTm | | | | | |
| rgLnNMSXX 60 C. * | 1 | 1 | 0.2497758 | 222.7130 | <.0001 |
| maxTmp | | | | | |
| flipError * maxTmp | 1 | 1 | 0.0724958 | 64.6410 | <.0001 |
| DuplexTm * maxTmp | 1 | 1 | 0.0403392 | 35.9685 | <.0001 |
| MaxSubSeqTm * | 1 | 1 | 0.1432244 | 127.7062 | <.0001 |
| maxTmp | | | | | |
| maxTmp * maxTmp | 1 | 1 | 0.0422151 | 37.6411 | <.0001 |
| rgLnNMSXX 60 C. * | 1 | 1 | 0.1583963 | 141.2343 | <.0001 |
| HomLogS2B | | | | | |
| flipError * | 1 | 1 | 0.0473319 | 42.2035 | <.0001 |
| HomLogS2B | | | | | |
| DuplexTm * | 1 | 1 | 0.0238086 | 21.2290 | <.0001 |
| HomLogS2B | | | | | |

In conclusion, the above analysis of variance studies showed that chromosome X probe performance as represented in LogRatio70 trends was most correlated with the rgLnNMSXX6070 values, as compared to the calculated Score of the probes and the signal intensity values at 60° C. Since the rgLnNMSXX6070 values are derived from the differences in signal values from probes hybridized at 60° C. and the same probes hybridized at 70° C., these values are related to the kinetics of the probe-target interactions at the different hybridization stringencies, and the correlation of the results of using methods described herein has been optimally validated and implemented using best statistics practice. Therefore, selection of probes according to these techniques should provide good leverage for probe design for all microarray platforms.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, algorithm, sample, experiment, process, process step or steps, to the objective, spirit and scope of the present invention. All such modifications are intended to be within the scope of the claims appended hereto.

That which is claimed is:

1. A method of selecting probes for design of a chemical array said method comprising:
providing a first set of candidate probes for hybridization with a sample at a first hybridization stringency and a second set of candidate probes identical to the first set for hybridization with the sample at a second hybridization stringency;
hybridizing the first set with the sample at a first hybridization stringency;
hybridizing the second set with the sample at a second hybridization stringency higher than the first hybridization stringency;
calculating the relative change in signal extracted from a probe in the first set relative to the same probe in the second set and repeating the calculation step for each of a plurality of other probes in the first set and same probes in the second set, respectively; and
eliminating at least the probe having the highest calculated relative change in signal between the first and second hybridization stringencies.

2. The method of claim 1, wherein the first and second hybridization stringencies differ by hybridization temperature.

3. The method of claim 1, further comprising adding new candidate probes that were not present in the first and second sets to replace the at least one probe eliminated from each set, and repeating the steps of claim 1.

4. The method of claim 3, further comprising repeating iterations of replacements of new probes and repeating steps for a predetermined number of iterations or until a number of probes eliminated in an iteration is less than a predetermined number, and selecting the set of probes resulting after the second-to-last iteration of said eliminating step for use on a chemical array.

5. The method of claim 1, further comprising removing scanner offset values from the signals extracted from the probes prior to said calculating step.

6. The method of claim 5, further comprising converting the signal values having the scanner offset values removed to natural log signal values.

7. The method of claim 1, wherein the probes are feature extracted by two-channel feature extraction, and wherein the sample hybridized to the probes is a mixture of the sample labeled with a first label and the sample labeled with a second label.

8. The method of claim 6, wherein the probes are feature extracted by two-channel feature extraction, and wherein the sample hybridized to the probes is a mixture of the sample labeled with a first label and the sample labeled with a second label, said method further comprising calculating a mean signal of the natural log signals from the probe for the first labeled sample and the second labeled sample, for each probe.

9. A method of identifying relative degrees of non-specific binding of probes hybridized with a sample, said method comprising:
providing a first set of probes for hybridization with a sample at a first hybridization stringency and a second set of probes identical to the first set for hybridization with the sample at a second hybridization stringency;
hybridizing the first set with the sample at a first hybridization stringency;
hybridizing the second set with the sample at a second hybridization stringency higher than the first hybridization stringency;
calculating the relative change in signal extracted from a probe in the first set relative to the same probe in the second set and repeating the calculation step for each of a plurality of other probes in the first set and same probes in the second set, respectively; and
ranking the probes by degree of non-specific binding, wherein the probe having the highest calculated relative change in signal between the first and second hybridization stringencies is ranked highest.

10. The method of claim 9, wherein the first and second hybridization stringencies differ by hybridization temperature.

11. The method of claim 10, further comprising removing scanner offset values from the signals extracted from the probes prior to said calculating step.

12. The method of claim 11, further comprising converting the signal values having the scanner offset values removed to natural log signal values.

13. The method of claim 10, wherein the probes are feature extracted by two-channel feature extraction, and wherein the sample hybridized to the probes is a mixture of the sample labeled with a first label and the sample labeled with a second label.

14. The method of claim 12, wherein the probes are feature extracted by two-channel feature extraction, and wherein the sample hybridized to the probes is a mixture of the sample labeled with a first label and the sample labeled with a second label, said method further comprising calculating a mean signal of the natural log signals from the probe for the first labeled sample and the second labeled sample, for each probe.

15. A system for identifying relative degrees of non-specific binding of probes hybridized with a sample, wherein a first set of probes are hybridized with the a sample at a first hybridization stringency and a second set of probes identical to the first set are hybridized with the sample at a second hybridization stringency, said system comprising:
a processor; and
instructions executable by said processor for calculating the relative change in signal extracted from a probe in the first set relative to the same probe in the second set and repeating the calculation step for each of a plurality of other probes in the first set and same probes in the second set, respectively; and
ranking the probes by degree of non-specific binding, wherein the probe having the highest calculated relative change in signal between the first and second hybridization stringencies is ranked highest.

16. A system for selecting probes for design of a chemical array, wherein a first set of probes are hybridized with the a sample at a first hybridization stringency and a second set of probes identical to the first set are hybridized with the sample at a second hybridization stringency, said system comprising:
a processor; and
instructions executable by said processor for calculating the relative change in signal extracted from a probe in the first set relative to the same probe in the second set and repeating the calculation step for each of a plurality of other probes in the first set and same probes in the second set, respectively; and
eliminating at least the probe having the highest calculated relative change in signal between the first and second hybridization stringencies.

17. A computer readable medium carrying one or more sequences of instructions for identifying relative degrees of non-specific binding of probes hybridized with a sample, wherein a first set of probes are hybridized with the a sample at a first hybridization stringency and a second set of probes identical to the first set are hybridized with the sample at a second hybridization stringency, and wherein execution of one or more sequences of instructions by one or more processors causes the one or more processors to perform the steps of:
calculating the relative change in signal extracted from a probe in the first set relative to the same probe in the second set and repeating the calculation step for each of a plurality of other probes in the first set and same probes in the second set, respectively; and
ranking the probes by degree of non-specific binding, wherein the probe having the highest calculated relative change in signal between the first and second hybridization stringencies is ranked highest.

18. A computer readable medium carrying one or more sequences of instructions for selecting probes for design of a chemical array, wherein a first set of probes are hybridized with the a sample at a first hybridization stringency and a second set of probes identical to the first set are hybridized with the sample at a second hybridization stringency, and wherein execution of one or more sequences of instructions by one or more processors causes the one or more processors to perform the steps of:
calculating a relative change in signal extracted from a probe in the first set relative to the same probe in the second set and repeating the calculation step for each of a plurality of other probes in the first set and same probes in the second set, respectively; and
eliminating at least the probe having the highest calculated relative change in signal between the first and second hybridization stringencies.

19. The computer readable medium of claim 18, wherein the first and second hybridization stringencies differ by hybridization temperature.

20. The computer readable medium of claim 18, wherein the following further steps are performed: adding new candidate probes that were not present in the first and second sets to replace the at least one probe eliminated from each set, and repeating the steps of claim 16.

21. The computer readable medium of claim 20, wherein the following further steps are performed: repeating iterations of replacements of new probes and repeating steps for a predetermined number of iterations or until a number of probes eliminated in an iteration is less than a predetermined number, and selecting the set of probes resulting after the last iteration of said eliminating step for use on a chemical array.

22. The computer readable medium of claim 18, wherein the following further steps is performed: removing scanner offset values from the signals extracted from the probes prior to said calculating step.

23. The computer readable medium of claim 20, wherein the following further step is performed: converting the signal values having the scanner offset values removed to natural log signal values.

24. The computer readable medium of claim 18, wherein the probes are feature extracted by two-channel feature extraction, and wherein the sample hybridized to the probes is a mixture of the sample labeled with a first label and the sample labeled with a second label.

25. The computer readable medium of claim 23, wherein the probes are feature extracted by two-channel feature extraction, and wherein the sample hybridized to the probes is a mixture of the sample labeled with a first label and the sample labeled with a second label, said method further comprising calculating a mean signal of the natural log signals from the probe for the first labeled sample and the second labeled sample, for each probe.

26. A chemical array comprising probes selected by the method of claim 1.

27. A kit useful for selecting probes to be used on a chemical array, said kit comprising:
at least two arrays each provided with the same probe set; and
instructions for carrying out the method of claim 1.
